(19) United States
(12) Patent Application Publication
(10) Pub. No.: US 2010/0209496 A1
(43) Pub. Date: Aug. 19, 2010

(54) LIQUID AND SEMI-SOLID PHARMACEUTICAL FORMULATIONS FOR ORAL ADMINISTRATION OF A SUBSTITUTED AMIDE

(76) Inventors: Eleni Dokou, Harleysville, PA (US); Brian Farrer, Maplewood, NJ (US); Eric Kemp, Hatfield, PA (US); Lee Klein, Collegeville, PA (US)

Correspondence Address: MERCK, P O BOX 2000, RAHWAY, NJ, 07065-0907, US

(21) Appl. No.: 12/439,896

(22) PCT Filed: Aug. 31, 2007
(86) PCT No.: PCT/US07/19121

§ 371 Date: Mar. 4, 2009

Related U.S. Application Data
(60) Provisional application No. 60/842,576, filed on Sep. 6, 2006.
(60) Provisional application No. 60/855,188, filed on Oct. 30, 2006.

(51) Int. Cl. A61K 9/66; A61K 31/40

(52) U.S. Cl. 424/455; 514/416

(57) ABSTRACT

3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one (Compound I) has surprisingly improved solubility, bioavailability and stability in an ampiphilic vehicle that can include propylene glycol, a propylene glycol sparing agent, an antioxidant, or a mixture thereof. In one embodiment of the present invention is a solution comprising the active or a pharmaceutically acceptable salt or solvate thereof, and an amphiphilic agent, said amphiphilic agent being a fatty acid ester of glycerol, propylene glycol or sorbitol.

### BACKGROUND OF THE INVENTION

The compound 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one (Compound I), described in WO 2005/073191, is an NK-1 receptor antagonist.

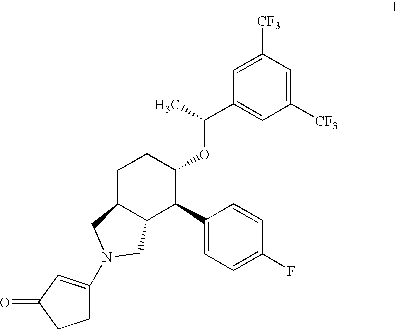

This invention relates to formulations of Compound I and pharmaceutically acceptable salts and solvates thereof for use in mammals, especially humans, especially encapsulated formulations, including hard and soft gelatin capsules, which formulations provide increased concentrations of Compound I for absorption; hence higher bioavailability.

The pharmaceutical industry is faced with the challenge of developing formulations for an increasing number of active molecules that possess low aqueous solubility and/or intestinal epithelial permeability. In some cases, as in the case of Compound I, acceptable bioavailability can not be readily achieved across the range of useful doses by means of traditional tablet or capsule formulations. An alternative dosage form for compounds with high lipid solubility is a lipid-based liquid-filled capsule (LFC). Such formulations have exhibited enhanced oral bioavailability and increased the interest in the potential of lipid-based formulations for oral administration. The exact mechanisms responsible for the enhanced bioavailability of poorly water soluble compounds are difficult to elucidate, but lipid-based formulations primarily increase exposure by overcoming the slow dissolution step from a solid dosage form (Pouton, C. W., Europ. J. Pharm. Sciences, 11 Suppl. 2 (2000) S93-S98). Additionally, these formulations may also enhance permeability (Aungst, B. J., J. Pharm. Sciences, 89:4 (2000) 429-442).

Therefore, there remains a need to develop stable oral formulations of Compound I that would maximize exposure and reduce potential variability in absorption due to the food effect. A formulation that permits the presentation of larger doses per capsule would also be a desirable result. This invention provides pharmaceutical compositions that are liquid solutions, semisolids, suspensions, and (oil-in-water) emulsions of Compound I, said solutions being orally administrable. The solutions or dispersions may be administered, for example, as fill in encapsulated dosage forms such as liquid filled and sealed hard gelatin capsules or soft gelatin capsules containing plasticizers, such as glycerin and sorbitol. Compound I can not be readily dissolved at relevant concentrations in many lipophilic vehicles, as further described and discussed below, such as digestible oils, cosolvents and surfactants.

### SUMMARY OF THE INVENTION

The present invention relates to pharmaceutical compositions for the oral administration of 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one (Compound I), a compound with low aqueous solubility (<0.2 μg/mL).

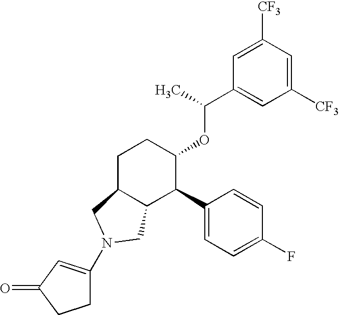

Compound I is an NK-1 receptor antagonist. Compositions of the present invention comprising Compound I are useful in the treatment of diseases mediated by the antagonism of the NK-1 receptor.

### BRIEF DESCRIPTION OF THE DRAWINGS

FIG. 1: Mean plasma concentration versus time after an oral dose administration of 40 mg of Compound of Formula I in liquid filled capsules.

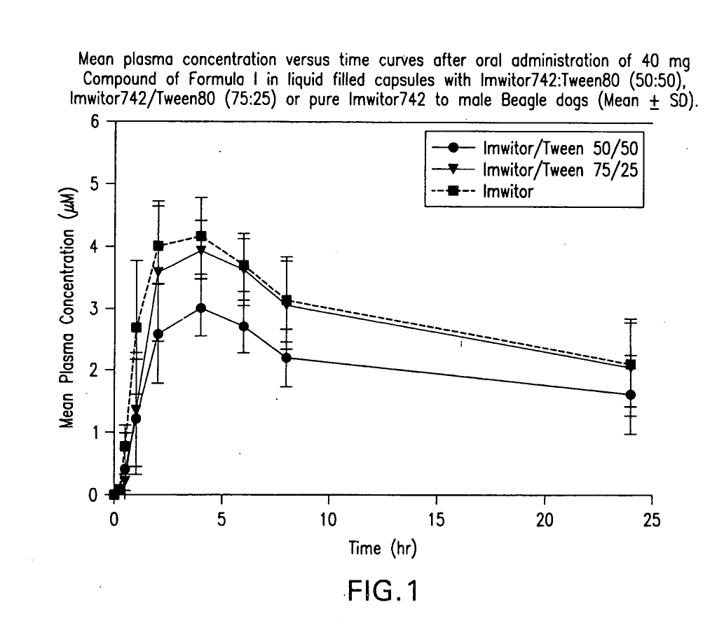

### DETAILED DESCRIPTION OF THE INVENTION

The present invention relates to pharmaceutical compositions for the oral administration of N-[1S,2S]-3-(4-chlorophenyl)-2-(3-cyanophenyl)-1-methylpropyl]-2-methyl-2-{[5-trifluoromethyl]pyridine-2-yl}oxy}propanamide, (Compound I) a compound with low aqueous solubility (<0.2 μg/mL). The invention is a formulation which increases the bioavailability, as determined by drug plasma levels, while maintaining chemical integrity of Compound I in the formulation upon storage. When dosed as a crystalline solid, the compound was found to be poorly bioavailable in dogs and rats. It has been found that bioavailability is increased dramatically by using a liquid-filled capsule dosage form in which the compound is in solution in certain combinations of liquid and semi-solid excipients. However, the low solubility and/or chemical instability of Compound I in the overwhelming majority of vehicles typically used in the formulation of low aqueous solubility compounds necessitated extensive research and ingenuity to arrive at the surprising invention disclosed herein.

In one embodiment, the invention is directed to a solution comprising the active agent 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one or a pharmaceutically acceptable salt or solvate thereof, and an amphiphilic agent, said amphiphilic agent being a fatty acid ester of glycerol, propylene glycol or sorbitol.

Within this embodiment, there is a genus wherein said amphiphilic agent consisting essentially of mono- and di-glycerides of C8 to C12 saturated fatty acids and mixtures thereof.

As one of skill in the art will appreciate, commercially produced amphiphilic agents can contain modest amounts of non-amphiphilic agents such as triglycerides, including those containing C8 and C10 fatty acids. For purposes of this specification, amphiphilic agents are defined to include such commercial products. On the other hand, such non-amphiphilic agents are not material to the invention (i.e. they do not increase the solubility of Compound of Formula I in the liquid formulation.). For example, the compound of Formula I, is not sufficiently soluble in Miglyol 812, to provide a fully efficacious amount of solubilized compound in a 500 mg capsule.

Within this embodiment there is another genus wherein the amphiphilic agent is selected from the group consisting of CAPMUL MCM, CAPMUL MCM 8, CAPMUL MCM 10, IMWITOR 988, IMWITOR 742, IMWITOR 642, IMWITOR 308, CAPRYOL PGMC, CAPRYOL 90, LAUROGLYCOL, CAPTEX 200, CRILL 1, CRILL 4, PECEOL and MAISINE.

Within this genus, there is a sub-genus wherein the amphiphilic agent is IMWITOR 742.

Within this embodiment, there is another genus comprising:

(a) 50% or less by weight active agent; and

(b) 50% or greater by weight amphiphilic agent.

Within this genus, there is a sub-genus comprising:

(a) 0.03% to 5.0% by weight active agent; and

(b) 95% to 99.9% r by weight amphiphilic agent.

Within this genus, there is another sub-genus comprising:

(a) 0.10% to 5.0% by weight active agent; and

(b) 95% to 99.9% r by weight amphiphilic agent.

Within this sub-genus, there is a class comprising:

(a) 0.5% to 4% by weight active agent; and

(b) 96% to 99.5% by weight amphiphilic agent.

Within this embodiment, there is another genus further comprising propylene glycol.

Within this genus, there is a sub-genus further comprising an anti-oxidant, wherein the weight of the antioxidant is less than 1% of the solution.

Within this genus, there is a sub-genus wherein the weight ratio of amphiphilic agent to propylene glycol is greater than 1 to 1.

Within this sub-genus, there is a class wherein the weight ratio of amphiphilic agent to propylene glycol is between 1 to 1 and 10 to 1.

Within this genus, there is another sub-genus further comprising a propylene glycol sparing agent selected from glycerin or ethanol or mixtures thereof.

Within this sub-genus, there is a class comprising:

(a) 0.03 to 5.0% by weight active agent;

(b) 50 to 94.9% by weight amphiphilic agent;

(c) 5 to 49.9% by weight propylene glycol;

(d) 0 to 20% by weight glycerine; and

(e) 0 to 20% by weight of ethanol.

Within this sub-genus, there is another class comprising:

(a) 0.10 to 5.0% by weight active agent;

(b) 50 to 94.9% by weight amphiphilic agent;

(c) 5 to 49.9% by weight propylene glycol;

(d) 0 to 20% by weight glycerine; and

(e) 0 to 20% by weight of ethanol.

Within this class, there is a sub-class comprising:

(a) 0.5 to 4.0% by weight active agent;

(b) 50 to 89.5% by weight amphophillic agent;

(c) 10 to 49.5% by weight propylene glycol;

(d) 0 to 10% by weight glycerine; and

(e) 0 to 5% by weight of ethanol.

Within this embodiment there is a genus further comprising an anti-oxidant, wherein the weight of the antioxidant is less than 1% of the solution.

Within this genus, there is a sub-genus wherein the weight of the antioxidant is less than 0.2% of the solution.

Within this sub-genus, there is a class wherein the weight of the antioxidant is between 0.05 and 0.15% of the solution.

Within this class, there is a sub-class wherein the anti-oxidant is selected from butylated hydroxyanisole, butylated hydroxytoluene, propyl gallate and sodium sulfite and mixtures thereof.

In a second embodiment the invention is directed to a capsule containing a solution according to the first embodiment, wherein the solution comprises: 0.25 to 25 mg of the active agent 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one or a pharmaceutically acceptable salt or solvate thereof, and 50-950 mg of an amphiphilic agent, said amphophillic agent consisting essentially of mono- and di-glycerides of C8 to C12 saturated fatty acids and mixtures thereof.

Within the second embodiment, is a genus wherein the solution comprises: 0.25 to 25 mg of the active agent 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one or a pharmaceutically acceptable salt or solvate thereof, and 775 to 800 mg of an amphiphilic agent, said amphophillic agent consisting essentially of mono- and di-glycerides of C8 to C12 saturated fatty acids and mixtures thereof.

Within this genus, there is a sub-genus wherein the amphiphilic agent is selected from the group consisting of CAPMUL MCM, CAPMUL MCM 8, CAPMUL MCM 10, IMWITOR 988, IMWITOR 742, and IMWITOR 308.

Within this sub-genus, there is a class wherein the amphiphilic agent is IMWITOR 742.

Within this genus, is a sub-genus wherein the solution further comprises 0-50% propylene glycol.

Within this sub-genus, is class wherein the solution comprises 20-40% propylene glycol.

Within the sub-genuses comprising propylene glycol, is a class wherein the solution further comprising 0-20% of a propylene glycol sparing agent selected from glycerin or ethanol or mixtures thereof.

Within the sub-genuses comprising propylene glycol, there is a class further containing 10-20% of a propylene glycol sparing agent selected from glycerin or ethanol or mixtures thereof.

Within the sub-genuses comprising propylene glycol, there is a class further containing 0-1% of an antioxidant.

Within this class, is a sub-class wherein the antioxidant is selected from butylated hydroxyanisole, butylated hydroxytoluene, propyl gallate and sodium sulfite and mixtures thereof.

Within the sub-genuses containing propylene glycol, there is a class further containing 0.01-0.2% of an antioxidant.

Within this class is a sub-class containing 0.1% butylated hydroxyanisole.

Within the second embodiment, there is a genus wherein the amount of 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one is selected from: 0.05 mg, 0.1 mg, 0.25 mg, 0.5 mg, 1 mg, 2 mg, 2.5 mg, 3 mg, 4 mg, 5 mg, 6 mg, 7 mg, 7.5 mg, 8 mg, 9 mg, 10 mg, 15 mg, 20 mg and 25 mg.

Within this genus, there is a sub-genus wherein the amount of 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one is selected from: 1 mg, 2 mg, 2.5 mg, 3 mg, 4 mg, 5 mg, 6 mg, 7 mg, 7.5 mg, 8 mg, 9 mg, and 10 mg.

Within this sub-genus, there is a class wherein the solution comprises: 50-500 mg of an amphiphilic agent, said amphiphilic agent consisting essentially of mono- and di-glycerides of C8 to C12 saturated fatty acids and mixtures thereof.

Within the second embodiment, there is a genus wherein the capsule is selected from a soft-gelatin capsule and a hard gelatin capsule.

Within this genus, there is a sub-genus which is an oral pharmaceutical composition.

Within the first embodiment, there is a genus for use in treating a condition selected from: urinary incontinence, urinary frequency, overeactive bladder and urinary frequency disorders in a human in need of such treatment.

Reference herein is made to a soft gelatin capsule, “softgel” being an abbreviation for soft gelatin capsules. It is understood that when reference is made to the term “softgel” alone, it shall be understood that the invention applies equally to all types of gelatin and non-gelatin capsules, regardless of hardness, softness, and so forth. In one embodiment of the present invention, the soft gelatin capsule contains plasticizers, such as glycerin and sorbitol. Colorant may be added to the gel mixture prior to encapsulation to produce soft gelatin capsules of the desired hue.

The pre-concentrate can be self-emulsifying, self-microemulsifying, or non-emulsifying. The term “self-emulsifying” refers to a formulation which, when diluted by a factor of at least 100 by water or other aqueous medium and gently mixed, yields an opaque, stable oil/water emulsion with a mean droplet diameter less than about 5 microns, but greater than 250 nm, and which is generally polydisperse. The term “self-microemulsifying” refers to a pre-concentrate which, upon at least 100× dilution with an aqueous medium and gentle mixing, yields a non-opaque, stable oil/water emulsion with an average droplet size of about 1 micron or less, said average particle size preferably being less than 250 nm. The particle size is primarily unimodal. Both self-emulsifying, self-microemulsifying, and non-emulsifying formulations are encompassed by the present invention.

The composition can be formulated as a fill encapsulated in a gelatin capsule of appropriate gelatin composition, a hard gelatin capsule with an appropriate seal, a non-gelatin capsule such as a hydroxypropyl methylcellulose capsule, or an oral liquid or emulsion by methods commonly employed in the art. In one embodiment of the present invention, the fill is encapsulated in a sealed hard gelatin capsule or a soft gelatin capsule containing plasticizers, such as glycerin and sorbitol. In one class of this embodiment, the hard gelatin capsule is sealed by band sealing using a gelatin ribbon, or using LEMS (Liquid Encapsulation Microspray Sealing Technology, i.e., spraying with a hydroalcoholic solution to locally melt and then let the capsules dry to fuse/seal the gelatin capsule pieces). The fill is prepared by mixing the excipients and Compound I with heating if required.

Oral delivery of Compound I is particularly difficult because its aqueous solubility is extremely low, typically being less than 0.2 μg/mL. Achieving therapeutic drug levels in the blood by oral dosing of practical quantities of a drug generally requires a large enhancement in drug concentrations in the gastrointestinal fluid and a resulting large enhancement in bioavailability. The formulations of this invention will be administered in such an amount that an effective dose of Compound I is administered to the patient. The amount of Compound I will generally be known or determined by the attending physician. Thus, the amount or volume of preconcentrate administered will be determined by the amount of Compound I prescribed and/or otherwise desired as a dose and the solubility of the Compound I in the preconcentrate. In general, an effective dose for Compound I is from 0.05 mg or 0.25 mg to about 25 mg per day, in single or divided doses; preferably from about 1.0 mg to about 10 mg per day, in single or divided doses. For oral administration, the compositions are preferably provided in the form of liquid- or semi-solid-filled capsules containing from 0.05 to 25 mg or 0.25 to 25 mg, preferably 0.05, 0.1, 0.25, 0.5, 1, 2, 2.5, 3, 4, 5, 6, 7, 7.5, 8, 9, 10, 15, 20 or 25, most preferably 1, 5, or 10 milligrams of the active ingredient for the symptomatic adjustment of the dosage to the patient to be treated.

The compositions of the invention are pre-concentrates which are generally administered orally, in soft or hard gelatin capsules, gelatin encapsulation technology being well known to the pharmaceutical arts. Such pre-concentrates can also be administered in aqueous oral emulsions by adding the pre-concentrate to water or other aqueous liquid (e.g., soda). They can be mixed with an aqueous liquid and sold as pre-formed emulsions, or added to food such as ice cream.

Compositions of the present invention comprising Compound I are useful in the prevention and treatment of a wide variety of clinical conditions which are characterized by the presence of an excess of tachykinin, in particular substance P, activity. Thus, for example, an excess of tachykinin, and in particular substance P, activity is implicated in a variety of disorders of the central nervous system. Such disorders include mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders; schizophrenia and other psychotic disorders, for example, schizophreniform disorders, schizoaffective disorders, delusional disorders, brief psychotic disorders, shared psychotic disorders and psychotic disorders with delusions or hallucinations; delerium, dementia, and amnestic and other cognitive or neurodegenerative disorders, such as Alzheimer's disease, senile dementia, dementia of the Alzheimer's type, vascular dementia, and other dementias, for example, due to HIV disease, head trauma, Parkinson's disease, Huntington's disease, Pick's disease, Creutzfeldt-Jakob disease, or due to multiple aetiologies; Parkinson's disease and other extra-pyramidal movement disorders such as medication-induced movement disorders, for example, neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremour; substance-related disorders arising from the use of alcohol, amphetamines (or amphetamine-like substances), caffeine, cannabis, cocaine, hallucinogens, inhalants and aerosol propellants, nicotine, opioids, phenylglycidine derivatives, sedatives, hypnotics, and anxiolytics, which substance-related disorders include dependence and abuse, intoxication, withdrawal, intoxication delerium, withdrawal delerium, persisting dementia, psychotic disorders, mood disorders, anxiety disorders, sexual dysfunction and sleep disorders; epilepsy; Down's syndrome; demyelinating diseases such as MS and ALS and other neuropathological disorders such as peripheral neuropathy, for example diabetic and chemotherapy-induced neuropathy, and postherpetic neuralgia, trigeminal neuralgia, segmental or intercostal neuralgia and other neuralgias; and cerebral vascular disorders due to acute or chronic cerebrovascular damage such as cerebral infarction, subarachnoid haemorrhage or cerebral oedema.

Tachykinin, and in particular substance P, activity is also involved in nociception and pain. Compound I of the present invention will therefore be of use in the prevention or treatment of diseases and conditions in which pain predominates, including soft tissue and peripheral damage, such as acute trauma, osteoarthritis, rheumatoid arthritis, musculo-skeletal pain, particularly after trauma, spinal pain, myofascial pain syndromes, headache, episiotomy pain, and burns; deep and visceral pain, such as heart pain, muscle pain, eye pain, orofacial pain, for example, odontalgia, abdominal pain, gynaecological pain, for example, dysmenorrhoea, and labour pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stem damage; low back pain; sciatica; ankylosing spondylitis, gout; and scar pain.

Tachykinin, and in particular substance P, antagonists may also be of use in the treatment of respiratory diseases, particularly those associated with excess mucus secretion, such as chronic obstructive airways disease, bronchopneumonia, chronic bronchitis, cystic fibrosis and asthma, adult respiratory distress syndrome, and bronchospasm; inflammatory diseases such as inflammatory bowel disease, psoriasis, fibrositis, osteoarthritis, rheumatoid arthritis, pruritis and sunburn; allergies such as eczema and rhinitis; hypersensitivity disorders such as poison ivy; ophthalmic diseases such as conjunctivitis, vernal conjunctivitis, and the like; ophthalmic conditions associated with cell proliferation such as proliferative vitreoretinopathy; cutaneous diseases such as contact dermatitis, atopic dermatitis, urticaria, and other eczematoid dermatitis. Tachykinin, and in particular substance P, antagonists may also be of use in the treatment of neoplasms, including breast tumours, neuroganglioblastomas and small cell carcinomas such as small cell lung cancer.

Tachykinin, and in particular substance P, antagonists may also be of use in the treatment of gastrointestinal (GI) disorders, including inflammatory disorders and diseases of the GI tract such as gastritis, gastroduodenal ulcers, gastric carcinomas, gastric lymphomas, disorders associated with the neuronal control of viscera, ulcerative colitis, Crohn's disease, irritable bowel syndrome and emesis, including acute, delayed or anticipatory emesis such as emesis induced by chemotherapy, radiation, toxins, viral or bacterial infections, pregnancy, vestibular disorders, for example, motion sickness, vertigo, dizziness and Meniere's disease, surgery, migraine, variations in intercranial pressure, gastro-oesophageal reflux disease, acid indigestion, over indulgence in food or drink, acid stomach, waterbrash or regurgitation, heartburn, for example, episodic, nocturnal or meal-induced heartburn, and dyspepsia.

Tachykinin, and in particular substance P, antagonists may also be of use in the treatment of a variety of other conditions including stress related somatic disorders; reflex sympathetic dystrophy such as shoulder/hand syndrome; adverse immunological reactions such as rejection of transplanted tissues and disorders related to immune enhancement or suppression such as systemic lupus erythematosus; plasma extravasation resulting from cytokine chemotherapy, disorders of bladder function such as cystitis, bladder detrusor hyper-reflexia, frequent urination and urinary incontinence, including the prevention or treatment of overactive bladder with symptoms of urge urinary incontinence, urgency, and frequency; fibrosing and collagen diseases such as scleroderma and eosinophilic fascioliasis; disorders of blood flow caused by vasodilation and vasospastic diseases such as angina, vascular headache, migraine and Reynaud's disease; and pain or nociception attributable to or associated with any of the foregoing conditions, especially the transmission of pain in migraine. The compounds of the present invention are also of value in the treatment of a combination of the above conditions, in particular in the treatment of combined post-operative pain and post-operative nausea and vomiting.

Compound I of the present invention is particularly useful in the prevention or treatment of emesis, including acute, delayed or anticipatory emesis, such as emesis induced by chemotherapy, radiation, toxins, pregnancy, vestibular disorders, motion, surgery, migraine, and variations in intercranial pressure. For example, the compounds of the present invention are of use optionally in combination with other antiemetic agents for the prevention of acute and delayed nausea and vomiting associated with initial and repeat courses of moderate or highly emetogenic cancer chemotherapy, including high-dose cisplatin. Most especially, the compounds of the present invention are of use in the treatment of emesis induced by antineoplastic (cytotoxic) agents, including those routinely used in cancer chemotherapy, and emesis induced by other pharmacological agents, for example, rolipram. Examples of such chemotherapeutic agents include alkylating agents, for example, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; and cytotoxic antibiotics. Particular examples of chemotherapeutic agents are described, for instance, by D. J. Stewart in Nausea and Vomiting: Recent Research and Clinical Advances, Eds. J. Kucharczyk et al, CRC Press Inc., Boca Raton, Fla., USA (1991) pages 177-203, especially page 188. Commonly used chemotherapeutic agents include cisplatin, dacarbazine (DTIC), dactinomycin, mechlorethamine, streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin and chlorambucil [R. J. Gralla et al in Cancer Treatment Reports (1984) 68(1), 163-172]. A further aspect of the present invention comprises the use of a compound of the present invention for achieving a chronobiologic (circadian rhythm phase-shifting) effect and alleviating circadian rhythm disorders in a mammal. The present invention is further directed to the use of a compound of the present invention for blocking the phase-shifting effects of light in a mammal.

The present invention is further directed to the use of a compound of the present invention or a pharmaceutically acceptable salt thereof, for enhancing or improving sleep quality as well as preventing and treating sleep disorders and sleep disturbances in a mammal. In particular, the present invention provides a method for enhancing or improving sleep quality by increasing sleep efficiency and augmenting sleep maintenance. In addition, the present invention provides a method for preventing and treating sleep disorders and sleep disturbances in a mammal which comprising the administration of a compound of the present invention or a pharmaceutically acceptable salt thereof. The present invention is useful for the treatment of sleep disorders, including Disorders of Initiating and Maintaining Sleep (insomnias) (“DIMS”) which can arise from psychophysiological causes, as a consequence of psychiatric disorders (particularly related to anxiety), from drugs and alcohol use and abuse (particularly during withdrawal stages), childhood onset DIMS, nocturnal myoclonus, fibromyalgia, muscle pain, sleep apnea and restless legs and non specific REM disturbances as seen in ageing.

The terms “administration of” and or “administering a” compound should be understood to mean providing the composition of the invention to the individual in need of treatment.

The administration of the composition of the present invention to practice the present methods of therapy is carried out by administering an effective amount of the compound of structural formula I to the patient in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

Representative experimental procedures are provided below. These are exemplary only and should not be construed as being limitations on the novel compositions and processes of this invention.

TABLE 1
Compound I Form I Solubility in Various Liquid Vehicles
(values are in mg/g of formulation)

| | Room | | | |
|---|---|---|---|---|
| Solvent | Temp | 30° C. | 35° C. | 65° C. |
| Aqueous Systems | | | | |
| Water | <0.0002 | | | |
| pH 1.3 | 0.001 | | | |
| pH 2.2 to 10.1 | <0.0002 | | | |
| 0.5% Methylcellulose | 0.001 | | | |
| Oils | | | | |
| Soybean Oil | <0.1 | | | |
| Olive Oil | <0.1 | | | |
| Corn Oil | <0.1 | | | |
| Miglyol 812 | 0.4 | | | |
| Other Liquid Vehicles | | | | |
| Tween 20 | 2 | | | |
| Tween 80 | 1 | | | |
| PEG 400 | 2 | | | |
| Propylene Glycol | 5 | | 8 | |
| Cremophor EL | 2 | | | |
| Labrasol | 7 | | 11 | |
| Imwitor 742 | >6 | 43 | 46 | |
| Imwitor 642 | | | 78 | |
| Imwitor 308 | | | 58 | |
| Imwitor 988 | | | 54 | |
| Capryol PGMC | >6 | 29 | | |
| Glycerin | <0.5 | | | |
| Ethanol | 58 | | | |
| Capmul MCM C-8 | | | 53 | |
| Capmul PG-8 | | | 50 | |
| Capmul PG-12 | | | 25 | |
| Acconon MC-8 | | | <1 | |
| Captex 200P | | | 2 | |
| Peceol | | | 19 | |
| Labrafil M2125CS | 5 | | 4 | |
| Labrafil M1944CS | | | 4 | |
| Gelucire 44/14 | | | | 4 |
| Addition of Acid/Base | | | | |
| PEG 400:water (92:8) | 1.0 | | | |
| PEG 400:water (92:8) + 1 eq | 1.3 | | | |
| NaOH | | | | |
| PEG 400:water (92:8) + 1 eq HCl | 24 | | | |
| 1:1 Imwitor:Tween + 1 eq HCl | >57 | | | |

### Example 1

Stability of 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one, (Compound I) in Various Liquid Vehicles

TABLE 2
Compound I Chemical Stability in Various Liquid Vehicles (Values are % claim at 40°
C. with respect to −20° C. control, 1 mg Compound I/g vehicle unless otherwise stated)

| Formulation | 1 wk | 2 wk | 4 wk | 5 wk | 8 wk | 9.5 wk | 18 wk | 32.5 wk |
|---|---|---|---|---|---|---|---|---|
| Vehicle | | | | | | | | |
| PEG 400 | | | | | | | | |
| Imwitor 742 | 99.8 | | | 98.8 | | 99.8 | | 97.0 |
| Capmul MCM C8 | 99.0 | | | | | | | 78.1 |
| Capmul PG-8 | 99.7 | | | | | | | |
| Vehicle Mixture | | | | | | | | |
| 1:1 Imwitor 742:Tween 80 | 95.2 | | 90.4 | 83.8 | | | 73.7 | |
| 9:1 Imwitor 742:Tween 80 | 99.6 | | | | | 96.4 | | |
| 6:4 Imwitor 742:Propylene Glycol | 100.2 | 100.6 | 100.0 | | 100.6 | | | |
| Added Antioxidant | | | | | | | | |
| 1:1 Imwitor 742:Tween 80 | | | | | | | | |
| +0.1% BHA | 98.4 | | 96.1 | | | | | |
| +0.1% BHT | | | | | | | | |
| +0.1% Propyl Gallate | 99.6 | | 97.7 | | | | 93.1 | |
| +0.1% α-tocopherol | 86.7 | | 71.1 | | | | | |
| +0.1% Ascorbic Acid Palmitate | 94.2 | | 89.4 | | | | | |
| +0.05% PG + 0.05% BHA | 102.2 | | | | | | 94.7 | |
| +0.05% PG + 0.05% BHT | 99.4 | | | | | | 93.7 | |
| +0.05% PG + 0.05% NaSO₃ | 98.5 | | | | | | | |
| Added Acid (at 10 mg/g) | | | | | | | | |
| Imwitor 742:Tween 80 | | | | | | | | |
| +0.75 eq HCl | 95.2 | | | | | | | |
| +0.75 eq Sulfuric Acid | 82.9 | | | | | | | |
| +0.75 eq Phosphoric Acid | 96.4 | | | | | | | |
| +0.75 eq Benzenesulfonic Acid | 99.1* | | | | | | | |
| PEG 400 + 0.75 eq HCl | 60.3 | | | | | | | |
| Propylene Glycol + 0.75 eq HCl | 95.2 | | | | | | | |
| Imwitor 742 + 0.75 eq HCl | 100.6 | | | | | | | |

*Athough there is only a small loss of Compound 1 in the samples with Benzenesulfonic acid, degradation products were observed in the benzenesulfonic acid sample and none were observed in the Imwitor 742 samples. In addition, benzenesulfonic acid did not completely dissolve in Imwitor:Tween.

### Example 2

An example of the procedure used to prepare capsule dosage forms for Compound I is given below:

1. The mono- and diglycerides excipient (e.g., IMWITOR 742) is melted at an appropriate temperature.
2. Antioxidant is added to the mixture and dissolved.
3. The Compound I is added to the mixture and dissolved.
4. The mixture is filled into hard gelatin capsules or suitably formulated soft gelatin capsules. For hard gelatin capsules, the filled capsules are sealed appropriately.

### Example 3

Mean pharmacokinetic parameters after oral administration of 50 mg Compound I in liquid-filled gelatin capsules to male Rhesus Monkeys (mean+/−SD).

Fasted male Rhesus monkeys (New Iberia, La.) were used for the monkey studies. All animals were fasted for 16 hours prior to dosing. They were housed in an AAALAC-accredited facility in accordance with USDA guidelines. After an overnight fast, capsules were administered to the monkeys orally via gavage tube and were followed immediately by 20 mL of water. Each formulation was tested in three monkeys (n=3). Water was returned at 1 hour after dosing and food was returned at 4 hours after dosing. Blood was drawn via venipuncture using a 21 g butterfly needle inserted into the saphenous vein at pre-dose and 15, 30, 60, 120, 240, 360, 480, and 1440 minutes after dosing. The plasma was separated by centrifugation (15 minutes at 2500 rpm) and kept frozen at −70° C. until analysis by LC/MS/MS.

A sensitive analytical method using liquid chromatography/electrospray ionization tandem mass spectrometry (LC/ESI-MS/MS) for the quantitation of Compound I in monkey plasma was developed and validated. The method employed a protein precipitation procedure using acetonitrile to isolate Compound I from the biological matrix. An analog of Compound I, N-[3-(4-fluoro-phenyl)-2-(3-cyano-phenyl)-1-methylpropyl]-2-(5-trifluoromethyl-2-pyridyloxy)-2-methylpropanamide; was used as the internal standard. Reconstituted extracts were ionized by a TurboIonSpray interface and analyzed in the selected reaction monitoring (SRM) mode. Chromatography was performed on a 100×2 mm, 5 μm, AQUASIL C8 column using 75:25 acetonitrile and 25 mM ammonium formate, pH 3.0. Under these conditions, no interference was observed for either Compound I or the internal standard from the endogenous components of dog plasma. The assay had a lower limit of quantitation (LOQ) of 1 ng/mL in plasma for Compound I based on 0.1-mL aliquots of plasma. The standard curve range was from 1 to 5000 ng/mL. The analysis time was 5.0 minutes per sample.

Area under the curve (AUC₀₋₂₄), mean and standard deviation of AUC, observed maximum plasma concentration (Cₘₐₓ), and time of Cₘₐₓ (Tₘₐₓ) were calculated with WinNonLin v3.1.

TABLE 3
Mean pharmacokinetic parameters after oral administration
of 40 mg Compound 1 in liquid filled capsules with
Imwitor742:Tween80 (50:50), Imwitor742/Tween80 (75:25) or
pure Imwitor742 to male Beagle dogs (Mean ± SD).

| | Imwitor/Tween | Imwitor/Tween | |
|---|---|---|---|
| | (50:50) | (75:25) | Imwitor |
| AUC₀₋₂₄ | 48.95 ± 11.53 | 65.41 ± 14.68 | 69.02 ± 14.23 |
| (μM · hr) | | | |
| Cₘₐₓ | 3.04 ± 0.44 | 4.06 ± 0.49 | 4.22 ± 0.54 |
| (μM) | | | |
| Tₘₐₓ | 3.67 ± 0.82 | 3.33 ± 1.03 | 4.67 ± 1.63 |
| (hr) | | | |

### Examples 4A and 4B

### Example 4A

### Manufacturing Process—Fill Compounding

The mono- and diglycerides excipient (e.g., IMWITOR 742) was melted at an appropriate temperature (40° C.±5° C.). The antioxidant (Butylated Hydroxyanisole) was added to the mono and diglycerides in an appropriate vessel and the materials were mixed until dissolved (solution homogeneous), at 35° C.±5° C.

Compound I was added to the IMWITOR 742, and the contents were mixed at 35° C.±5° C. until Compound I was dissolved. The solution was deaerated under vacuum until visual examination revealed that all air was removed (at least 15 min). The solution was filtered through a 35 micron mesh filter. The solution was kept at 30° C. until encapsulation. The fill formulation composition is given in Table 4 below.

### Manufacturing Process—Encapsulation:

The fill mixture and the gelatin mixture were compounded separately. These materials were then fed into the encapsulation machine. The gelatin used was acid bone and lime bone bovine gelatin, containing glycerin and sorbitol as plasticizers and red and yellow iron oxides and titanium dioxide as colorants.

To encapsulate the fill solution, the gelatin formulation was cast into sheets on two cooled rollers. These sheets were passed through a series of rolls where a food grade lubricant was applied. The sheets were then fed through the rotary die rolls where the softgel was formed. As the lower edge of the softgel was formed, a reciprocating pump injected the fill solution into the center of the softgel after which the upper edge of the die came together to seal the softgel. The newly formed softgels were dislodged from the sheet and pneumatically conveyed to a tumble dryer where they stayed for 45-60 minutes. Upon exiting the dryer, the softgels were spread on trays and placed in a drying tunnel (low humidity chamber) and dried. Upon completion of the drying process, the softgels were visually inspected for defects. Subsequently, the capsules were sized to remove oversize and undersized capsules and polished.

TABLE 4
Fill formulation composition and batch information for preparation
of soft gelatin liquid filled capsules (per batch basis)

| | Composition | Weight per | Weight per |
|---|---|---|---|
| Material | % wt | unit (mg) | batch (g) |
| Imwitor 742 | 98.90 | 395.60 | 1186.80 |
| BHA | 0.10 | 0.40 | 1.20 |
| Compound I | 1.00 | 4.00 | 12.00 |
| Total Batch | 100.00 | 400.00 | 1200.00 |

### Example 4B

The IMWITOR 742 was melted at an appropriate temperature (40° C.±5° C.). Imwitor 742 was added to a preheated vessel (35° C.±5° C.) to avoid solidification. The Butylated Hydroxyanisole is also added to the vessel and mixed until homogeneous (dissolved) for at least 15 min. Compound I was slowly added to the mixture and dissolved. The materials were mixed using high and/or low shear mixing at 35° C.±5° C. for at least 15 min or until Compound I was fully dissolved. Once Compound I dissolution was confirmed visually, in process samples were taken and they were visually inspected for the presence of particulates and analyzed by HPLC to verify that the solution concentration reached the target value. The fill solution was charged to the encapsulator (Shionogi F40) product hopper for encapsulation. The liquid formulation was dispensed into the size 0 or 00, white, opaque hard gelatin capsules (Licap CAPSUGEL capsules, containing gelatin and titanium dioxide) to a target fill weight of 400 or 800 mg. The filled capsules were transferred to a Shionogi S40 capsule band sealer and they were sealed by placing a small band of gelatin on the interface between the capsule shell body and cap. The banding solution consists of gelatin, Polysorbate 80 water and colorant if necessary. After banding the capsules were placed onto drying trays lined with drying paper and were stored for a minimum of 4 hours prior to being visually inspected for leaking. The acceptable capsules were weight sorted using a Shionogi Capsule Inspection Machine CWI-80. The finished capsules were then packaged into appropriate containers.

The following Tables provides illustrative examples of the preparation of 0.25 mg, 1 mg, 4 mg and 25 mg hard shell capsules. Soft shell capsules may be prepared with identical ingredients, and in fact, has been prepared, in the case of the 4 mg capsule.

TABLE 5
Fill formulation composition for the preparation of hard gelatin liquid filled capsules (per capsule basis)

| | | Unit Strength | | | | | | | |
|---|---|---|---|---|---|---|---|---|---|
| Components | Function | 0.25 mg | | 1 mg | | 4 mg | | 25 mg | |
| Capsule Fill | | | | | | | | | |
| Compound I per capsule | Active | 0.25 | mg | 1.0 | mg | 4.0 | mg | 25 | mg |
| Mono-& Diglycerides (Imwitor 742) | Solubilizer | 399.35 | mg | 398.6 | mg | 395.6 | mg | 774.2 | mg |
| Butylated Hydroxyanisole | Antioxidant | 0.40 | mg | 0.40 | mg | 0.40 | mg | 0.8 | mg |
| Capsule Shell | | | | | | | | | |
| #0 White Opaque Hard Gelatin Capsule^{‡} | Capsule Shell | 96 | mg* | 96 | mg* | 96 | mg* | — | |
| #00 White Opaque Hard Gelatin Capsule^{‡} | Capsule Shell | — | | — | | — | | 113 | mg* |
| Gelatin** | Banding | — | | — | | — | | — | |
| | component | | | | | | | | |
| Polysorbate 80** | Banding | — | | — | | — | | — | |
| | component | | | | | | | | |
| Total Fill | | 400 | mg | 400 | mg | 400 | mg | 800 | mg |

^{‡}Capsules are provided by Capsugel and contain gelatin and titanium dioxide.
*approximate weight of empty capsule shell
**As needed to seal the capsule shells

TABLE 6
Drug product (Compound I) batch formulas

| | | Unit Strength | | | | | |
|---|---|---|---|---|---|---|---|
| Components | Function | 0.25 mg | | 1 mg | | 4 mg | |
| Capsule Fill | | | | | | | |
| Compound I | Active | 5.00 | g | 15.3 | g | 680.0 | g |
| Mono-& Diglycerides (Imwitor 742) | Solubilizer | 7987 | g | 6079 | g | 67252 | g |
| Butylated Hydroxyanisole | Antioxidant | 8.00 | g | 6.1 | g | 68.0 | g |
| Capsule Shell | | | | | | | |
| #0 White Opaque Hard Gelatin Capsule | Capsule Shell | 20000 | units | 15250 | units | 170000 | units |
| Total Fill | | 8000 | g | 6100 | g | 68000 | g |

| | | Unit Strength | |
|---|---|---|---|
| Components | Function | 25 mg | |
| Capsule Fill | | | |
| Compound I | Active | 175.0 | g |
| Mono-& Diglycerides (Imwitor 742) | Solubilizer | 5419.4 | g |
| Butylated Hydroxyanisole | Antioxidant | 5.6 | g |
| Capsule Shell | | | |
| #00 White Opaque Hard Gelatin Capsule | Capsule Shell | 7000 | units |
| Total Fill | | 5600 | g |

While the invention has been described and illustrated with reference to certain particular embodiments thereof, those skilled in the art will appreciate that various changes, modifications and substitutions can be made therein without departing from the spirit and scope of the invention. It is intended, therefore, that the invention be defined by the scope of the claims which follow and that such claims be interpreted as broadly as is reasonable.

What is claimed is:

1. A solution comprising the active agent 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one or a pharmaceutically acceptable salt or solvate thereof, and an amphiphilic agent, said amphiphilic agent being a fatty acid ester of glycerol, propylene glycol or sorbitol.

2. A solution according to claim 1, wherein said amphiphilic agent consisting essentially of mono- and di-glycerides of C8 to C12 saturated fatty acids and mixtures thereof.

3. A solution according to claim 1 wherein the amphiphilic agent is selected from the group consisting of CAPMUL MCM, CAPMUL MCM 8, CAPMUL MCM 10, IMWITOR 988, IMWITOR 742, IMWITOR 642, IMWITOR 308, CAPRYOL PGMC, CAPRYOL 90, LAUROGLYCOL, CAPTEX 200, CRILL 1, CRILL 4, PECEOL and MAISINE.

4. A solution according to claim 3 wherein the amphiphilic agent is IMWITOR 742.

5. A solution according to claim 1 comprising:
(a) 50% or less by weight active agent; and
(b) 50% or greater by weight amphiphilic agent;

6. A solution according to claim 5 comprising:
(a) 0.03% to 5.0% by weight active agent; and
(b) 95% to 99.9% r by weight amphiphilic agent.

7. A solution according to claim 5 comprising:
(a) 0.10% to 5.0% by weight active agent; and
(b) 95% to 99.9% r by weight amphiphilic agent.

8. A solution according to claim 7 comprising:
(a) 0.5% to 4% by weight active agent; and
(b) 96% to 99.5% by weight amphiphilic agent.

9. A solution according to claim 1 further comprising propylene glycol.

10. A solution according to claim 9, wherein the weight ratio of amphiphilic agent to propylene glycol is greater than 1 to 1.

11. A solution according to claim 10 wherein the weight ratio of amphiphilic agent to propylene glycol is between 1 to 1 and 10 to 1.

12. A solution according to claim 9 further comprising a propylene glycol sparing agent selected from glycerin or ethanol or mixtures thereof.

13. A solution according to claim 12 comprising:
(a) 0.03 to 5.0% by weight active agent;
(b) 50 to 94.9% by weight amphiphilic agent;
(c) 5 to 49.9% by weight propylene glycol;
(d) 0 to 20% by weight glycerin; and
(e) 0 to 20% by weight of ethanol.

14. A solution according to claim 12 comprising:
(a) 0.10 to 5.0% by weight active agent;
(b) 50 to 94.9% by weight amphiphilic agent;
(c) 5 to 49.9% by weight propylene glycol;
(d) 0 to 20% by weight glycerin; and
(e) 0 to 20% by weight of ethanol.

15. A solution according to claim 14 comprising:
(a) 0.5 to 4.0% by weight active agent;
(b) 50 to 89.5% by weight amphophillic agent;
(c) 10 to 49.5% by weight propylene glycol;
(d) 0 to 10% by weight glycerine; and
(e) 0 to 5% by weight of ethanol.

16. A solution according to claim 1, claim 4 or claim 9 further comprising an anti-oxidant, wherein the weight of the antioxidant is less than 1% of the solution.

17. A solution according to claim 16, wherein the weight of the antioxidant is less than 0.2% of the solution.

18. A solution according to claim 17, wherein the weight of the antioxidant is between 0.05 and 0.15% of the solution.

19. A solution according to claim 16, wherein the anti-oxidant is selected from butylated hydroxyanisole, butylated hydroxytoluene, propyl gallate and sodium sulfite and mixtures thereof.

20. A capsule containing a solution according to claim 1, wherein the solution comprises:
0.05 to 25 mg of the active agent 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one or a pharmaceutically acceptable salt or solvate thereof, and 50-950 mg of an amphiphilic agent, said amphophillic agent consisting essentially of mono- and di-glycerides of C8 to C12 saturated fatty acids and mixtures thereof.

21. A capsule containing a solution according to claim 20, wherein the solution comprises:
0.25 to 25 mg of the active agent 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one or a pharmaceutically acceptable salt or solvate thereof, and 775 to 800 mg of an amphiphilic agent, said amphophillic agent consisting essentially of mono- and di-glycerides of C8 to C12 saturated fatty acids and mixtures thereof.

22. A capsule according to claim 21 wherein the amphiphilic agent is selected from the group consisting of CAPMUL MCM, CAPMUL MCM 8, CAPMUL MCM 10, IMWITOR 988, IMWITOR 742, and IMWITOR 308.

23. A capsule according to claim 22 wherein the amphiphilic agent is IMWITOR 742.

24. A capsule according to claim 22 wherein the solution further comprises 0-50% propylene glycol.

25. A capsule according to claim 24 wherein the solution comprises 20-40% propylene glycol.

26. A capsule according to claim 24 wherein the solution further comprises 0-20% of a propylene glycol sparing agent selected from glycerin or ethanol or mixtures thereof.

27. A capsule according to claim 26 wherein the solution comprises 10-20% of a propylene glycol sparing agent selected from glycerin or ethanol or mixtures thereof.

28. The capsule according to claim 24 wherein the solution further comprises 0-1% of an antioxidant.

29. The capsule according to claim 28 wherein the solution comprises 0.01-0.2% of an antioxidant.

30. The capsule according to claim 28, wherein the antioxidant is selected from butylated hydroxyanisole, butylated hydroxytoluene, propyl gallate and sodium sulfite and mixtures thereof.

31. A capsule according to claim 23 wherein the solution further comprises 0.1% butylated hydroxyanisole.

32. A capsule according to claim 20, wherein the amount of 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one is selected from: 0.05 mg, 0.1 mg, 0.25 mg, 0.5 mg, 1 mg, 2 mg, 2.5 mg, 3 mg, 4 mg, 5 mg, 6 mg, 7 mg, 7.5 mg, 8 mg, 9 mg, 10 mg, 15 mg, 20 mg and 25 mg.

33. A capsule according to claim 32, wherein the amount of 3-[(3aR,4R,5S,7aS)-5-{(1R)-1-[3,5-bis(Trifluoromethyl)phenyl]ethoxy}-4-(4-fluorophenyl)-octahydro-2H-isoindol-2-yl]cyclopent-2-en-1-one is selected from: 1 mg, 2 mg, 2.5 mg, 3 mg, 4 mg, 5 mg, 6 mg, 7 mg, 7.5 mg, 8 mg, 9 mg, and 10 mg.

34. A capsule containing a solution according to claim 33, wherein the solution comprises:
50-500 mg of an amphiphilic agent, said amphiphilic agent consisting essentially of mono- and di-glycerides of C8 to C12 saturated fatty acids and mixtures thereof.

35. The capsule according to claim 20, wherein the capsule is selected from a soft-gelatin capsule and a hard gelatin capsule.

36. A solution to claim 35 which is an oral pharmaceutical composition.

37. The solution according to claim 1 for use in treating a condition selected from: urinary incontinence, urinary frequency, overactive bladder and urinary frequency disorders in a human in need of such treatment.
